(19) United States
(12) Patent Application Publication
(10) Pub. No.: US 2026/0036547 A1
(43) Pub. Date: Feb. 5, 2026

(54) METHODS FOR DETECTING METABOLITES USING A MICROFLUIDIC-BASED CE-MS SYSTEM

(71) Applicant: ASTRAZENECA AB, Södertälje (SE)

(72) Inventors: Erik ALLMAN, Gaithersburg, MD (US); Sonja HESS, Gaithersburg, MD (US); Christina DI POTO, Gaithersburg, MD (US)

(21) Appl. No.: 19/099,577

(22) PCT Filed: Jul. 28, 2023
(86) PCT No.: PCT/EP2023/071033

§ 371 Date: Jan. 29, 2025

Related U.S. Application Data
(60) Provisional application No. 63/369,819, filed on Jul. 29, 2022.

(51) Int. Cl. G01N 27/447; G01N 33/50; H01J 49/00; H01J 49/16; H01J 49/26

(52) U.S. Cl. CPC G01N 27/44756; G01N 33/5005; H01J 49/0018; H01J 49/0031; H01J 49/167; H01J 49/26; G01N 2800/52; G01N 2800/7095

(57) ABSTRACT

The present disclosure relates to methods of detecting metabolites using a microfluidic capillary electrophoresis-mass spectrometry (CE-MS) system. The metabolites can be useful to diagnosis diseases or disorders, as well as to monitor therapeutic efficacy of compounds used to treat these diseases or disorders.

### FIELD OF THE DISCLOSURE

The present disclosure relates to methods of detecting metabolites using a capillary electrophoresis-mass spectrometry (CE-MS) system. The metabolites can be useful to diagnosis of diseases or disorders, including airway inflammation diseases, cough, heart diseases, eye diseases, neurodegenerative diseases, psychiatric diseases, neuropathic pain, chronic inflammatory diseases, metabolic diseases, or cancer, as well as to monitor therapeutic efficacy of compounds used to treat these diseases or disorders.

### BACKGROUND

Many metabolites are known to be associated with the etiology of disease. For example, aberrant nucleotide concentrations are associated with a number of diseases. Adenosine 5′-triphosphate (ATP), and specifically extracellular ATP (eATP), is not only involved in airway inflammation diseases and cough, but blocking its extracellular release has been shown to produce a therapeutic benefit. These data support the notion that eATP may be a key driver of symptoms and pathogenesis of airway disease. However, detection methods for eATP remain problematic.

ATP is a complex nucleoside triphosphate, consisting of the nitrogenous base adenine, a ribose sugar, and a triphosphate chain. It is readily catalyzed to ADP, AMP, CAMP, adenosine and other downstream metabolites such as inosine. Unfortunately, ATP and similar nucleotide analogues are poorly retained by traditional reversed-phase liquid-chromatography (RPLC); do not migrate in coated chip capillary electrophoresis applications; are unstable and subject to interconversion from enzymes, pH, and/or temperature; have multiple pKas; and are metal-sensitive analytes.

Historically, ion-pairing chromatography or passivation has provided a solution to separate challenging compounds, such as ATP, however they can be problematic for liquid chromatography/mass spectrometry (LC/MS) systems. Moreover, common ATP luminescence-based assays indirectly measure ATP levels through enzymatic degradation, while lacking a simultaneous readout for its analogues. Thus, analytical techniques for detecting these challenging metabolites remains underdeveloped.

### BRIEF SUMMARY

The present disclosure is directed to a method for detecting a metabolite of interest in a sample, comprising: (a) contacting a sample comprising one or more metabolites of interest with an uncoated capillary electrophoresis (CE) platform; (b) separating the metabolites by molecular weight and/or charge in one or more capillaries using CE; (c) eluting the metabolite from the one or more capillaries; and (d) detecting the eluted metabolite by mass spectrometry analysis.

The present disclosure is also directed to a method for detecting a metabolite of interest in a sample, comprising: (a) contacting a sample comprising one or more metabolites of interest with a capillary electrophoresis (CE) platform having a chemically-modified surface; (b) separating the metabolites by molecular weight and/or charge in one or more capillaries using CE; (c) eluting the metabolite from the one or more capillaries; and (d) detecting the eluted metabolite by mass spectrometry analysis.

In one aspect, the CE platform is a microchip-based system. In another aspect, the microchip-based CE platform integrates electrophoretic separation and electrospray ionization into a mass spectrometer.

In one aspect, the metabolite of interest is listed in Table 1. In another aspect, the metabolite of interest is an anionic metabolite. In another aspect, the metabolite is a nucleotide, nucleotide analog, or degradation product. In another aspect, the metabolite of interest is adenosine 5′-triphosphate (ATP).

In one aspect, the sample is a blood, plasma, cell, or lavage sample. In another aspect, the sample is a bronchoalveolar lavage fluid (BALF). In a further aspect, the sample comprises a chelating agent. In another aspect, the chelating agent is ethylenediaminetetraacetic acid (EDTA).

The present disclosure is also directed to a method of diagnosing a disease or disorder associated with aberrant nucleotide-dependent signaling in a subject, comprising: (a) contacting a sample from the subject with microchip-based capillary electrophoresis (CE) platform; (b) separating the adenosine 5′-triphosphate (ATP), ATP analogues and/or degradation products by molecular weight and/or charge in one or more capillaries using CE; and (c) eluting the ATP, ATP analogues and/or degradation products from the one or more capillaries; and (d) detecting the eluted ATP, ATP analogues and/or degradation products by mass spectrometry analysis; wherein the microchip-based CE platform integrates electrophoretic separation and electrospray ionization into a mass spectrometer; and wherein the presence of ATP, ATP analogues and/or degradation products is indicative of a disease or disorder associated with aberrant nucleotide-dependent signaling in the subject.

The present disclosure is also directed to a method of monitoring the therapeutic benefit of a compound on a disease or disorder associated with aberrant nucleotide-dependent signaling in a subject treated with the compound, comprising: (a) contacting a sample from the subject with microchip-based capillary electrophoresis (CE) platform; (b) separating the ATP, ATP analogues and/or degradation products by molecular weight and/or charge in one or more capillaries using CE; and (c) eluting the ATP, ATP analogues and/or degradation products from the one or more capillaries; and (d) detecting the eluted ATP, ATP analogues and/or degradation products by mass spectrometry analysis; wherein the microchip-based CE platform integrates electrophoretic separation and electrospray ionization into a mass spectrometer, and wherein the presence of ATP, ATP analogues and/or degradation products is indicative of a disease or disorder associated with aberrant nucleotide-dependent signaling in the subject.

In one aspect, the disease or disorder is associated with increased levels of extracellular ATP (eATP). In another aspect, the disease or disorder is an airway inflammation disease, cough, heart disease, eye disease, neurodegenerative disease, psychiatric disease, neuropathic pain, a chronic inflammatory disease, metabolic disease, or cancer. In another aspect, the cough is chronic idiopathic cough. In another aspect, the chronic inflammatory disease is systemic lupus erythematosus or Crohn's disease.

In one aspect, the sample is a blood, plasma, cell, or lavage sample. In another aspect, the sample is a BALF. In another aspect, the sample comprises a chelating agent. In another aspect, the chelating agent is ethylenediaminetetraacetic acid (EDTA).

In one aspect, the microchip is a ZipChip™. In another aspect, the microchip comprises a chemically-modified surface. In another aspect, the microchip does not comprise a surface modification.

In one aspect, the method further comprises adjusting the pH of the background electrolyte (BGE) relative to the metabolite of interest prior to mass spectrometry analysis.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 shows the capillary electrophoresis parameters used with the ZipChip™ platform.

FIG. 2 shows the mass spectrometer parameters used with the ZipChip™ platform.

FIG. 3 shows an electropherogram of the separation of ATP and metabolite species.

### DETAILED DESCRIPTION

### I. General Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer and fraction within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

### II. Detection Methods

The present disclosure relates to methods of detecting metabolites of interest in a sample. In some aspects, the metabolite of interest is associated with a disease state. Therefore, in some aspects, the disclosure relates to methods of diagnosing a disease or disorder, or monitoring therapeutic efficacy of a compound against a disease or disorder by detecting a metabolite of interest.

In one aspect, the disclosure relates to a method for detecting a metabolite of interest in a sample, comprising: (a) contacting a sample comprising one or more metabolites of interest with an uncoated capillary electrophoresis (CE) platform; (b) separating the metabolites by molecular weight and/or charge in one or more capillaries using CE; (c) eluting the metabolite from the one or more capillaries; and (d) detecting the eluted metabolite by mass spectrometry (MS) analysis. In some aspects, the method comprises an uncoated microchip-based CE platform. In another aspect, the microchip-based CE platform integrates electrophoretic separation and electrospray ionization into a mass spectrometer.

CE/MS systems combine capillary electrophoresis and mass spectrometry to separate and analyze samples. A CE/MS system works by first separating the ionic components of a sample by applying voltage to the sample. The ions will move through the capillary at different rates due to charge and frictional forces. The separated sample is then sprayed into the mass spectrometer which produces spectra. The spectra used to identify the individual components of the sample. In one aspect, the microchip contains the capillary. In another aspect, the CE is performed separately from the microchip.

The term “sample,” as used herein, refers to a mixture of components that includes at least a metabolite of interest, such as ATP, that is subjected to manipulation in accordance with the methods of the invention, including, for example, separating, analyzing, extracting, or profiling.

A “metabolite” as used herein refers to endogenous compounds such as amino acids, lipids, sugars, organic acids, etc., which are routinely being formed during anabolism or catabolism processes. Metabolites can have a multitude of functions, including energy conversion, signaling, epigenetic influence, and cofactor activity, but their presence can also be associated with human diseases or disorders. Exemplary metabolites of the present disclosure are found in Table 1.

“Subject” as used herein refers to a mammal, for example a dog, a cat, a horse, or a rabbit. In certain embodiments, the subject is a non-human primate, for example a monkey, chimpanzee, or gorilla. In certain embodiments, the subject is a human. “Subject” can be used interchangeably with “patient.”

As used herein, a “therapeutic benefit” relates to amelioration of symptoms or slowing of disease progression.

The terms “analysis” or “analyzing,” as used herein, are used interchangeably and refer to any of the various methods of separating, detecting, isolating, purifying, solubilizing, and/or characterizing metabolites of interest.

“Detect” and “detection” have their standard meaning, and are intended to encompass detection including the presence or absence, measurement, and/or characterization of a metabolite of interest, for example, ATP.

TABLE 1
Exemplary metabolites

| Metabolite | Disease Association |
|---|---|
| 1,11-undecanedicarboxylate | Colorectal cancer |
| 1,3,7-trimethyluric acid | Colorectal cancer; Asthma |
| 1,3-dihydro-(2h)-indol-2-one | Colorectal cancer |
| 10-hydroxydecanoate | N/A |
| 12,13-dihome | N/A |
| 16-hydroxy hexadecanoic acid | N/A |
| 1-methyladenosine | Cholangiocarcinoma; Colorectal cancer; Hepatocellular |
| | carcinoma; Ovarian cancer; Stomach cancer; Leukemia; |
| | Cerebrotendinous xanthomatosis; Uremia; Kidney |
| | disease |
| 1-methylhistamine | Ulcerative colitis; Crohn's disease; Mastocytosis; |
| | Alzheimer's disease; Dermal fibroproliferative disorder; |
| | Schizophrenia |
| 1-methylhistidine | Eosinophilic esophagitis; Diabetes mellitus type 2; |
| | Propionic acidemia; Early preeclampsia; Late-onset |
| | preeclampsia; Alzheimer's disease; Obesity; Pregnancy; |
| | Kidney disease |
| 1-methylnicotinamide | Autosomal dominant polycystic kidney disease; Lung |
| | Cancer; Eosinophilic esophagitis; Cirrhosis; Pellagra |
| 1-methylxanthine | Colorectal cancer; Asthma |
| 2,3-diaminopropionic acid | N/A |
| 2,6-pyridinedicarboxylic acid | N/A |
| 2-aminoheptanoate | Colorectal cancer |
| 2-aminoisobutyrate | Ulcerative colitis; Crohn's disease |
| 2-deoxy-d-glucose | N/A |
| 2-furoylglycine | Colorectal cancer; Eosinophilic esophagitis |
| 2-hydroxy-4- | N/A |
| (methylthio)butanoate | |
| 2-hydroxymyristic acid | Colorectal cancer |
| 2-hydroxypyridine | N/A |
| 2-methylbutyrylglycine | Autosomal dominant polycystic kidney disease; |
| | Glutaric aciduria II; Isovaleric acidemia; Propionic |
| | acidemia; Short/branched chain acyl-CoA |
| | dehydrogenase deficiency |
| 2-phenylglycine | N/A |
| 2-quinolinecarboxylate | N/A |
| 3-alpha,11-beta,17,21- | N/A |
| tetrahydroxy-5-beta-pregnan-20- | |
| one | |
| 3-carboxy-4-methyl-5-propyl-2- | Colorectal cancer; Uremia |
| furanpropionic acid | |
| 3-hydroxy-3-methylglutarate | Colorectal cancer; 3-Hydroxy-3-methylglutaryl-CoA |
| | lyase deficiency |
| 3-hydroxyanthranilate | Stroke; Nicotinamide Adenine Dinucleotide Deficiency |
| 3-hydroxydodecanoic acid | Fatty Acid Oxidation disorder; Nonalcoholic fatty liver |
| | disease |
| 3-hydroxyhippuric acid | Colorectal cancer; Eosinophilic esophagitis |
| 3-hydroxypicolinic acid | N/A |
| 3-hydroxytetradecanoic acid | Colorectal cancer |
| 3-methoxytyramine | Brunner Syndrome; Parkinson's disease |
| 3-methoxytyrosine | Aromatic L-amino acid decarboxylase deficiency; |
| | Pyridoxamine 5-prime-phosphate oxidase deficiency; |
| | Sepiapterin reductase deficiency; Epilepsy, early-onset, |
| | vitamin B6-dependent |
| 3-methylcrotonylglycine | 3-Hydroxy-3-methylglutaryl-CoA lyase deficiency; |
| | Biotinidase deficiency; Propionic acidemia |
| 3-methylhistidine | Colorectal cancer; Eosinophilic esophagitis; Diabetes |
| | mellitus type 2; Propionic acidemia; Early |
| | preeclampsia; Alzheimer's disease; Obesity; Pregnancy; |
| | Kidney disease |
| 3-nitro-l-tyrosine | Oxidative stress; Hypoxic-ischemic encephalopathy; |
| | Head injury; Meningitis |
| 3-oxindole-3-acetate | Colorectal cancer |
| 3-phenylpropionylglycine | N/A |
| 3-sulfinoalanine | Methotrexate treatment |
| 4-acetamidobutanoate | Colorectal cancer |
| 4-acetamidophenylglucuronide | Beta-thalassemia |
| 4-guanidinobutanoate | Colorectal cancer; Cirrhosis |
| 4-hydroxy-3-methoxyphenylglycol | N/A |
| 4-hydroxyhippuric acid | Colorectal cancer; Eosinophilic esophagitis |
| 4-hydroxy-l-glutamic acid | N/A |
| 4-imidazoleacetate | N/A |
| 4-pyridoxate | Colorectal cancer; Eosinophilic esophagitis; Ulcerative |
| | colitis; Crohn's disease; Iron deficiency |
| 5-acetylamino-6-amino-3- | Colorectal cancer |
| methyluracil | |
| 5-acetylamino-6-formylamino-3- | N/A |
| methyluracil | |
| 5-aminolevulinate | Eosinophilic esophagitis; Protoporphyria, |
| | Erythropoietic; Acute intermittent porphyria |
| 5′-deoxyadenosine | N/A |
| 5-hydroxy-l-tryptophan | Colorectal cancer; Ulcerative colitis; Crohn's disease; |
| | Aromatic L-amino acid decarboxylase deficiency |
| 5-keto-d-gluconate | Colorectal cancer |
| 5-methoxytryptophan | N/A |
| 5-methylcytosine | N/A |
| 5′-methylthioadenosine | Colorectal cancer |
| 6-hydroxynicotinate | Autosomal dominant polycystic kidney disease; |
| | Colorectal cancer; Prostate cancer; Diabetes mellitus |
| | type 2 |
| 6-oxopiperidine-2-carboxylic acid | Colorectal cancer |
| 7-ketodeoxycholate | Colorectal cancer |
| 7-methylguanine | Colorectal cancer |
| 7-methylxanthine | Colorectal cancer; Asthma |
| 8-hydroxyoctanoate | Colorectal cancer |
| adenine | Colorectal cancer; Eosinophilic esophagitis; Ulcerative |
| | colitis; Crohn's disease; Alzheimer's disease; |
| | Meningitis; Thymidine treatment; Tuberculous |
| | meningitis; Frontotemporal dementia; Lewy body |
| | disease |
| adenosine | Colorectal cancer; Eosinophilic esophagitis; Ulcerative |
| | colitis; Crohn's disease; Adenosine kinase deficiency; |
| | Adenylosuccinate lyase deficiency; Alzheimer's |
| | disease; Septic shock; Frontotemporal dementia; Lewy |
| | body disease; Irritable bowel syndrome |
| adenosine 2′,3′-cyclic phosphate | Colorectal cancer |
| Adenosine monophosphate | Colorectal cancer; Ulcerative colitis; Crohn's disease; |
| | Alzheimer's disease; Frontotemporal dementia; Lewy |
| | body disease; Metastatic melanoma |
| Adenosine triphosphate | Rachialgia; Stroke; Subarachnoid hemorrhage; |
| | Neuroinfection; Epilepsy; Chronic cough |
| ADP | Rachialgia; Stroke; Subarachnoid hemorrhage; |
| | Eosinophilic esophagitis; Neuroinfection; Epilepsy |
| aicar | N/A |
| ala-ala-ala | N/A |
| alanylglutamic acid | N/A |
| alanyl-glutamine | N/A |
| alanyl-histidine | Colorectal cancer |
| alanylmethionine | N/A |
| alanyl-proline | Colorectal cancer |
| alanyl-threonine | Colorectal cancer |
| alanyl-tryptophan | Colorectal cancer |
| alanyltyrosine | Colorectal cancer |
| alanylvaline | Colorectal cancer |
| allantoin | Colorectal cancer; Eosinophilic esophagitis; Ulcerative |
| | colitis; Crohn's disease; Meningitis; Schizophrenia; |
| | Chronic renal failure |
| allothreonine | Colorectal cancer; Prostate cancer |
| aminoadipate | Metastatic melanoma; Colorectal cancer; Eosinophilic |
| | esophagitis; Ulcerative colitis; Crohn's disease; 2- |
| | Ketoadipic acidemia; 2-Ketoadipic acidemia; |
| | Schizophrenia; Alpha-aminoadipic aciduria; Alpha- |
| | aminoadipic and alpha-ketoadipic aciduria |
| aniline-2-sulfonate | N/A |
| anserine | Colorectal cancer; Eosinophilic esophagitis; |
| | Alzheimer's disease |
| arginyl-phenylalanine | Colorectal cancer |
| asparaginyl-lysine | N/A |
| aspartate | Colorectal cancer; Crohn's disease; Autism; |
| | Schizophrenia; Irritable bowel syndrome; Periodontal |
| | disease; Colorectal cancer; Pancreatic cancer; |
| | Eosinophilic esophagitis; Growth hormone deficiency; |
| | Ulcerative colitis; Crohn's disease; Dicarboxylic |
| | aminoaciduria; Gout; Alzheimer's disease; Epilepsy; |
| | Perillyl alcohol administration for cancer treatment; |
| | Frontotemporal dementia; Lewy body disease; |
| | Schizophrenia; Irritable bowel syndrome |
| asymmetric dimethylarginine | Autosomal dominant polycystic kidney disease; |
| | Colorectal cancer; Essential hypertension; Uremia; |
| | Duchenne Muscular Dystrophy; Kidney disease |
| benzyl alcohol | Ulcerative colitis |
| beta-alanine | Periodontal disease; Colorectal cancer; Pancreatic |
| | cancer; Eosinophilic esophagitis; Ulcerative colitis; |
| | Crohn's disease; Dihydropyrimidine dehydrogenase |
| | deficiency; Gaba-transaminase deficiency; Hyper beta- |
| | alaninemia; Methylmalonate semialdehyde |
| | dehydrogenase deficiency; Alzheimer's disease; Perillyl |
| | alcohol administration for cancer treatment; |
| | Frontotemporal dementia; Lewy body disease |
| beta-glycerophosphate | Colorectal cancer |
| betaine | Periodontal disease; Colorectal cancer; Pancreatic |
| | cancer; Eosinophilic esophagitis; Ulcerative colitis; |
| | Crohn's disease; Hemodialysis; Argininosuccinic |
| | aciduria; Dimethylglycine Dehydrogenase Deficiency; |
| | Hypermethioninemia; Phenylketonuria; Propionic |
| | acidemia; Early preeclampsia; Late-onset preeclampsia; |
| | Alzheimer's disease; Continuous ambulatory peritoneal |
| | dialysis; Perillyl alcohol administration for cancer |
| | treatment; Pregnancy; Frontotemporal dementia; Lewy |
| | body disease; Schizophrenia; Chronic renal failure |
| beta-leucine | Vitamin B12 deficiency |
| biocytin | N/A |
| caffeine | Metastatic melanoma; Colorectal cancer; Eosinophilic |
| | esophagitis; Head injury |
| carnosine | Eosinophilic esophagitis; Ulcerative colitis; Crohn's |
| | disease; Carnosinuria; Alzheimer's disease |
| chenodeoxycholic acid glycine | Colorectal cancer; Biliary atresia |
| conjugate | |
| chiro-inositol | N/A |
| cinnamoylglycine | N/A |
| citrulline | Periodontal disease; Colorectal cancer; Pancreatic |
| | cancer; Eosinophilic esophagitis; Rheumatoid arthritis; |
| | Intestinal failure; Ulcerative colitis; Crohn's disease; |
| | Argininosuccinic aciduria; Citrullinemia type I; |
| | Citrullinemia type II, adult-onset; Citrullinemia type II, |
| | neonatal-onset; Fumarase deficiency; Gout; N- |
| | acetylglutamate synthetase deficiency; |
| | Phosphoenolpyruvate Carboxykinase Deficiency 1, |
| | Cytosolic; Myopathy, lactic acidosis, and sideroblastic |
| | anemia 1; Alzheimer's disease; Epilepsy; Cutis laxa, |
| | autosomal recessive, type IIIA; Perillyl alcohol |
| | administration for cancer treatment; Frontotemporal |
| | dementia; Lewy body disease; Schizophrenia; Pearson |
| | Syndrome |
| cortisol | Functional hypothalamic amenorrhea; Rheumatoid |
| | arthritis; ACTH deficiency, isolated; Adrenal |
| | hypoplasia; Adrenal insufficiency, congenital, with |
| | 46, XY sex reversal, partial or complete; 3-Hydroxy-3- |
| | methylglutaryl-CoA lyase deficiency; 3-Hydroxy-3- |
| | Methylglutaryl-CoA Synthase Deficiency; 3- |
| | Hydroxyacyl-CoA dehydrogenase deficiency; Adrenal |
| | hyperplasia, congenital, due to 3-beta-hydroxysteroid |
| | dehydrogenase 2 deficiency; Apparent |
| | mineralocorticoid excess; Aromatase deficiency; |
| | Congenital Adrenal Hyperplasia, due to 17- |
| | Hydroxylase-Deficiency; Glucocorticoid resistance; |
| | Leptin Deficiency or Dysfunction; Lipoid Congenital |
| | Adrenal Hyperplasia; Proprotein Convertase ⅓ |
| | Deficiency; Tic disorder; Anorexia nervosa; Benign |
| | gynecological diseases; Stress; Bipolar disorder; |
| | Schizophrenia; Corticosterone methyl oxidase I |
| | deficiency; Antley-Bixler syndrome with genital |
| | anomalies and disordered steroidogenesis; Bartter |
| | Syndrome, Type 2, Antenatal |
| cotinine | Smoking |
| creatinine | Colorectal cancer; Pancreatic cancer; Stomach cancer; |
| | 21-Hydroxylase deficiency; Ulcerative colitis; Crohn's |
| | disease; Hyperoxalemia; Canavan disease; |
| | Dimethylglycine Dehydrogenase Deficiency; Familial |
| | partial lipodystrophy; Isovaleric acidemia; Lesch- |
| | Nyhan syndrome; Partial lipodystrophy; |
| | Phenylketonuria; Phosphoribosylpyrophosphate |
| | Synthetase Superactivity; Propionic acidemia; |
| | Pseudohypoaldosteronism, type I, autosomal dominant; |
| | Tyrosinemia I; Early preeclampsia; Late-onset |
| | preeclampsia; Alzheimer's disease; Paraquat poisoning; |
| | Pregnancy; Frontotemporal dementia; Lewy body |
| | disease; Schizophrenia; Chronic renal failure; Primary |
| | hypomagnesemia; Cystic fibrosis; Bartter Syndrome, |
| | Type 2, Antenatal; Bartter Syndrome, Type 4A, |
| | Neonatal, with Sensorineural Deafness; Bartter |
| | Syndrome, Type 4B, Neonatal, With Sensorineural |
| | Deafness; Brown-Vialetto-Van Laere Syndrome 1; |
| | Cerebral creatine deficiency syndrome 2; |
| | Hypoparathyroidism-retardation-dysmorphism |
| | syndrome; Lipodystrophy, Congenital Generalized; |
| | Long-chain Fatty Acids, Defect in Transport of |
| cyclic amp | Idiopathic polyneuritis; Ulcerative colitis; Crohn's |
| | disease; Hypoxic-ischemic encephalopathy; Headache; |
| | Chronic renal failure |
| cyclic gmp | Headache |
| cystathionine | Eosinophilic esophagitis; Ulcerative colitis; Crohn's |
| | disease; Cystathioninuria; Folate deficiency; |
| | Hypermethioninemia; Vitamin B12 deficiency; |
| | Alzheimer's disease; Autism |
| cysteate | N/A |
| cysteine-s-sulfate | Eosinophilic esophagitis; Molybdenum cofactor |
| | deficiency; Sulfite oxidase deficiency, ISOLATED |
| cytidine | Colorectal cancer; Ulcerative colitis; Crohn's disease; |
| | Canavan disease; Alzheimer's disease; Frontotemporal |
| | dementia; Lewy body disease |
| cytidine 2′,3′-cyclic phosphate | N/A |
| cytosine | Colorectal cancer; Ulcerative colitis; Crohn's disease |
| daidzein | Colorectal cancer; Ileostomy |
| d-alanine | Propionic acidemia; Early preeclampsia; Late-onset |
| | preeclampsia; Pregnancy |
| decanoylcarnitine | Colorectal cancer; Eosinophilic esophagitis; Ulcerative |
| | colitis; Celiac disease; Crohn's disease; Glutaric |
| | aciduria II; Very Long Chain Acyl-CoA |
| | Dehydrogenase Deficiency; Obesity; Pregnancy |
| deoxyadenosine | Colorectal cancer; Eosinophilic esophagitis; Adenosine |
| | deaminase deficiency; Adenylosuccinate lyase |
| | deficiency |
| deoxycarnitine | Colorectal cancer |
| deoxycholic acid glycine | Colorectal cancer; Hepatocellular carcinoma |
| conjugate | |
| deoxycytidine | Colorectal cancer; Ulcerative colitis; Crohn's disease |
| deoxyguanosine | Colorectal cancer; Purine nucleoside phosphorylase |
| | deficiency |
| deoxyuridine-monophosphate | N/A |
| diaminopimelate | Colorectal cancer |
| diethanolamine | N/A |
| dihydrobiopterin | Colorectal cancer; Kidney disease |
| dl-2-aminooctanoic acid | Colorectal cancer |
| dl-methionine sulfoxide | Colorectal cancer; Eosinophilic esophagitis |
| dl-o-tyrosine | N/A |
| d-mannosamine | N/A |
| ectoine | N/A |
| epsilon-(gamma-glutamyl)lysine | Alzheimer's disease; Huntington's disease |
| equol | Ileostomy |
| ergothioneine | N/A |
| formiminoglutamic acid | Colorectal cancer; Glutamate formiminotransferase |
| | deficiency |
| fucose | Thyroid cancer; Colorectal cancer; Eosinophilic |
| | esophagitis |
| galactosamine | N/A |
| galactose 1-phosphate | Colorectal cancer |
| galacturonate | N/A |
| gamma-cehc | Colorectal cancer |
| gamma-glutamylglutamate | Colorectal cancer |
| gamma-glutamylglycine | N/A |
| gamma-glutamylhistidine | N/A |
| gamma-glutamylleucine | Colorectal cancer |
| gamma-glutamylvaline | Colorectal cancer |
| glucosamine | Colorectal cancer |
| glucosamine 6-sulfate | N/A |
| glucose 1-phosphate | Alzheimer's disease; Frontotemporal dementia; Lewy |
| | body disease |
| glucose 6-phosphate | Colorectal cancer; Eosinophilic esophagitis; |
| | Alzheimer's disease; Frontotemporal dementia; Lewy |
| | body disease |
| glucuronate | N/A |
| glutamate | Autosomal dominant polycystic kidney disease; |
| | Colorectal cancer; Ulcerative colitis; Crohn's disease; |
| | Gamma-glutamyltransferase deficiency; Alzheimer's |
| | disease; Pregnancy; Frontotemporal dementia; Lewy |
| | body disease; Schizophrenia; Irritable bowel syndrome; |
| | Autosomal dominant polycystic kidney disease; |
| | Periodontal disease; Colorectal cancer; Pancreatic |
| | cancer; Stomach cancer; Eosinophilic esophagitis; |
| | Ulcerative colitis; Crohn's disease; Anoxia; Sepsis; |
| | Leukemia; Dicarboxylic aminoaciduria; |
| | Lipoyltransferase 1 Deficiency; Heart failure; |
| | Alzheimer's disease; Epilepsy; Rett syndrome; Obesity; |
| | Perillyl alcohol administration for cancer treatment; |
| | Autism; Schizophrenia; Diverticular disease; Irritable |
| | bowel syndrome |
| glutamic acid gamma-methyl ester | N/A |
| glutamine | Periodontal disease; Colorectal cancer; Pancreatic |
| | cancer; Eosinophilic esophagitis; Ulcerative colitis; |
| | Crohn's disease; Leukemia; Carbamoyl Phosphate |
| | Synthetase Deficiency; Fumarase deficiency; |
| | Glutamine deficiency, congenital; Lipoyltransferase 1 |
| | Deficiency; Phosphoenolpyruvate Carboxykinase |
| | Deficiency 1, Cytosolic; Propionic acidemia; Early |
| | preeclampsia; Late-onset preeclampsia; Alzheimer's |
| | disease; Epilepsy; Obesity; Perillyl alcohol |
| | administration for cancer treatment; Pregnancy; |
| | Frontotemporal dementia; Lewy body disease; |
| | Schizophrenia; Irritable bowel syndrome |
| glutarylglycine | N/A |
| glycerol 3-phosphate | Colorectal cancer; Alzheimer's disease; Frontotemporal |
| | dementia; Lewy body disease |
| glycerophosphocholine | Periodontal disease; Kidney cancer; Pancreatic cancer; |
| | Multi-infarct dementia; Alzheimer's disease; Perillyl |
| | alcohol administration for cancer treatment |
| glycyl-asparagine | N/A |
| glycyl-aspartate | N/A |
| glycyl-threonine | Colorectal cancer |
| glycyltyrosine | Colorectal cancer |
| glycylvaline | Colorectal cancer |
| guanidinoacetate | N/A |
| guanidinosuccinate | Uremia; Chronic renal failure |
| guanidoacetic acid | Eosinophilic esophagitis; Ulcerative colitis; Crohn's |
| | disease; Uremia; Schizophrenia; Chronic renal failure; |
| | Cerebral creatine deficiency syndrome 1; Cerebral |
| | creatine deficiency syndrome 2; Cerebral creatine |
| | deficiency syndrome 3 |
| guanosine | Colorectal cancer; Eosinophilic esophagitis; Ulcerative |
| | colitis; Crohn's disease; Purine nucleoside |
| | phosphorylase deficiency |
| gulose | N/A |
| harman | N/A |
| hesperetin | Colorectal cancer |
| hexanoylglycine | Colorectal cancer; Propionic acidemia; Short Chain |
| | Acyl-Coa Dehydrogenase Deficiency; Ethylmalonic |
| | encephalopathy |
| hippurate | Colorectal cancer; Eosinophilic esophagitis; Diabetes |
| | mellitus type 1; Ulcerative colitis; Crohn's disease; |
| | Argininosuccinic aciduria; Phenylketonuria; Propionic |
| | acidemia; Tyrosinemia I; Uremia; Obesity; Paraquat |
| | poisoning; Schizophrenia |
| histamine | Nephrotic syndrome; Colorectal cancer; Eosinophilic |
| | esophagitis; Hemodialysis; Continuous ambulatory |
| | peritoneal dialysis; Kidney disease |
| histidinol | Prostate cancer |
| histidinyl-alanine | Colorectal cancer |
| histidinyl-isoleucine | Colorectal cancer |
| histidinyl-valine | Colorectal cancer |
| homocysteine | Colorectal cancer; Stroke; Hemodialysis; Sickle cell |
| | anemia; Transcobalamin II deficiency; |
| | Cystathioninuria; Dimethylglycine Dehydrogenase |
| | Deficiency; Homocystinuria; Sulfite oxidase |
| | deficiency, ISOLATED; Uremia; Dementia; Peripheral |
| | neuropathy; Progressive supranuclear palsy; |
| | Alzheimer's disease; Amyotrophic lateral sclerosis; |
| | Creutzfeldt-Jakob disease; Parkinson's disease; |
| | Continuous ambulatory peritoneal dialysis; Chronic |
| | renal failure; Methylenetetrahydrofolate reductase |
| | deficiency; Molybdenium co-factor deficiency; |
| | Molybdenum cofactor deficiency |
| homocysteine thiolactone | N/A |
| homogentisate | Eosinophilic esophagitis; Ulcerative colitis; Crohn's |
| | disease; Alkaptonuria |
| hydroxycotinine | Smoking |
| hydroxyisocaproic acid | Colorectal cancer; Eosinophilic esophagitis; Maple |
| | syrup urine disease |
| hyocholic acid | Colorectal cancer; Primary biliary cirrhosis |
| hypoxanthine | Autosomal dominant polycystic kidney disease; |
| | Degenerative disc disease; Periodontal disease; |
| | Colorectal cancer; Hepatocellular carcinoma; |
| | Pancreatic cancer; Eosinophilic esophagitis; |
| | Rheumatoid arthritis; Ulcerative colitis; Crohn's |
| | disease; Canavan disease; Lesch-Nyhan syndrome; |
| | Molybdenum cofactor deficiency; |
| | Phosphoribosylpyrophosphate Synthetase |
| | Superactivity; Sulfite oxidase deficiency, ISOLATED; |
| | Uremia; Xanthinuria type 1; Alzheimer's disease; |
| | Hydrocephalus; Perillyl alcohol administration for |
| | cancer treatment; Thymidine treatment; Frontotemporal |
| | dementia; Lewy body disease; Irritable bowel |
| | syndrome |
| iminodiacetic acid | N/A |
| indole-3-acetamide | N/A |
| indole-3-acetate | Appendicitis; Colorectal cancer; Eosinophilic |
| | esophagitis; Uremia; Tryptophanuria with dwarfism; |
| | Anorexia nervosa; Autism; Irritable bowel syndrome |
| indole-3-ethanol | N/A |
| indole-3-methyl acetate | N/A |
| indole-3-propionic acid | Colorectal cancer |
| indoleacetaldehyde | N/A |
| indoleacetic acid | Appendicitis; Colorectal cancer; Eosinophilic |
| | esophagitis; Uremia; Tryptophanuria with dwarfism; |
| | Anorexia nervosa; Autism; Irritable bowel syndrome |
| indoleacrylic acid | N/A |
| inosine | Degenerative disc disease; Colorectal cancer; |
| | Eosinophilic esophagitis; Ulcerative colitis; Crohn's |
| | disease; Coronary artery disease; Purine nucleoside |
| | phosphorylase deficiency; Canavan disease; Gout; |
| | Xanthinuria type 1; Critical illnesses; Septic shock; |
| | Thymidine treatment; Kidney disease; Irritable bowel |
| | syndrome |
| inosine-monophosphate | Febrile seizures; Crohn's disease; Ulcerative colitis |
| isobutyrate | Colorectal cancer; Eosinophilic esophagitis; Ulcerative |
| | colitis; Celiac disease; Crohn's disease; Nonalcoholic |
| | fatty liver disease; Early preeclampsia; Pregnancy; |
| | Irritable bowel syndrome |
| isoleucine | Periodontal disease; Colorectal cancer; Pancreatic |
| | cancer; Eosinophilic esophagitis; Ulcerative colitis; |
| | Crohn's disease; Branched-chain Keto Acid |
| | Dehydrogenase Kinase Deficiency; Leukemia; |
| | Dihydrolipoamide Dehydrogenase Deficiency; |
| | Lipoyltransferase 1 Deficiency; Maple syrup urine |
| | disease; Saccharopinuria; Early preeclampsia; Heart |
| | failure; Alzheimer's disease; Epilepsy; Perillyl alcohol |
| | administration for cancer treatment; Pregnancy; |
| | Frontotemporal dementia; Lewy body disease; Autism; |
| | Schizophrenia; Irritable bowel syndrome |
| isoleucyl-leucine | Colorectal cancer |
| isomaltose | Kidney disease |
| isoursodeoxycholic acid | Primary biliary cirrhosis |
| isovalerylcarnitine | Celiac disease; Isovaleric acidemia; Very Long Chain |
| | Acyl-CoA Dehydrogenase Deficiency |
| kynurenate | Colorectal cancer; Eosinophilic esophagitis; Ulcerative |
| | colitis; Crohn's disease; Malaria; Anemia; Uremia; |
| | Convulsion; Tuberculous meningitis; Schizophrenia |
| kynurenine | CNS tumors; Eosinophilic esophagitis; Ulcerative |
| | colitis; Crohn's disease; Nicotinamide Adenine |
| | Dinucleotide Deficiency; Tryptophanuria with |
| | dwarfism; CNS infections; Hydrocephalus; |
| | Intraventricular hemorrhage; Schizophrenia |
| kyotorphin | N/A |
| l,l-cyclo(leucylprolyl) | N/A |
| lactate | Diabetes mellitus type 2; D-Lactic Acidosis; |
| | Schizophrenia; D-Lactic Acidosis and Short Bowel |
| | Syndrome; Acute Infantile Liver Failure; Autosomal |
| | dominant polycystic kidney disease; Leigh Syndrome, |
| | French Canadian Type; Temporomandibular joint |
| | disorder; Colorectal cancer; Hepatic and biliary |
| | malignancies; Pancreatic cancer; Eosinophilic |
| | esophagitis; 2,4-dienoyl-CoA reductase deficiency; |
| | Diabetes mellitus type 1; Ulcerative colitis; Crohn's |
| | disease; Fructose intolerance, hereditary; Myopathy |
| | with lactic acidosis, hereditary; Anoxia; |
| | Hyperglycinemia, lactic acidosis, and seizures; |
| | Methylmalonic aciduria mitochondrial encephelopathy |
| | Leigh-like; 2-Ketoglutarate dehydrogenase complex |
| | deficiency; 2-Methyl-3-hydroxybutyryl-CoA |
| | dehydrogenase deficiency; 3-Hydroxy-3- |
| | methylglutaryl-CoA lyase deficiency; 3-Hydroxy-3- |
| | Methylglutaryl-CoA Synthase Deficiency; 3- |
| | Hydroxyacyl-CoA dehydrogenase deficiency; 3- |
| | Methylglutaconic Aciduria type VI; Amish lethal |
| | microcephaly; Coenzyme Q10 deficiency; Coenzyme |
| | Q10 deficiency, primary, 1; Combined oxidative |
| | phosphorylation deficiency 10; Cytochrome C oxidase |
| | deficiency; Dihydrolipoamide Dehydrogenase |
| | Deficiency; D-Lactic Acidosis; Fructose-1,6- |
| | diphosphatase deficiency; Lipoyltransferase 1 |
| | Deficiency; Mitochondrial complex I deficiency due to |
| | ACAD9 deficiency; Mitochondrial |
| | encephalomyopaththy with elevanted methylmalonic |
| | acid, SUCLA3; Mitochondrial Myopathy, Infantile, |
| | Transient; Mitochondrial phosphate carrier deficiency; |
| | Mitochondrial trifunctional protein deficiency; |
| | Mitochondrial-encephalopathy-lactic acidosis-stroke; |
| | Phosphoenolpyruvate Carboxykinase Deficiency 1, |
| | Cytosolic; Propionic acidemia; Pyruvate carboxylase |
| | deficiency; Pyruvate dehydrogenase deficiency; |
| | Pyruvate dehydrogenase phosphatase deficiency; |
| | Sulfite oxidase deficiency, ISOLATED; Early |
| | preeclampsia; Myoclonic epilepsy and ragged red fiber |
| | disease; Dementia; Late-onset preeclampsia; |
| | Alzheimer's disease; Epileptic encephalopathy, early |
| | infantile, 39; Leigh's syndrome, subacute necrotizing |
| | encephalopathy, SNE; Chronic progressive external |
| | ophthalmoplegia and Kearns-Sayre syndrome; Leber |
| | Optic Atrophy and Dystonia; Ethanol intoxication; |
| | Paraquat poisoning; Pregnancy; Frontotemporal |
| | dementia; Lewy body disease; Schizophrenia; Cerebral |
| | creatine deficiency syndrome 2; Deafness, |
| | Onychodystrophy, Osteodystrophy, Mental |
| | Retardation, and Seizures Syndrome; GRACILE |
| | syndrome; Infantile Liver Failure Syndrome 2; Irritable |
| | bowel syndrome; Pearson Syndrome; Sengers |
| | syndrome; Mitochondrial pyruvate carrier deficiency; |
| | Metabolic encephalomyopathic crises, recurrent, with |
| | rhabdomyolysis, cardiac arrhythmias, and |
| | neurodegeneration; Myopathy, lactic acidosis, and |
| | sideroblastic anemia 1 |
| lactose | Eosinophilic esophagitis; Lactose Intolerance |
| l-beta-aspartyl-l-glycine | N/A |
| l-beta-aspartyl-l-leucine | Colorectal cancer; Ulcerative colitis; Crohn's disease |
| l-carnitine | Periodontal disease; Colorectal cancer; Pancreatic |
| | cancer; Eosinophilic esophagitis; 2,4-dienoyl-CoA |
| | reductase deficiency; Diabetes mellitus type 2; 3- |
| | Hydroxy-3-methylglutaryl-CoA lyase deficiency; 3- |
| | Hydroxy-3-Methylglutaryl-CoA Synthase Deficiency; |
| | 3-Hydroxyacyl-CoA dehydrogenase deficiency; |
| | Carnitine palmitoyltransferase I deficiency; Carnitine |
| | transporter defect; primary systemic carnitine |
| | deficiency; L-2-Hydroxyglutaric aciduria; |
| | Oculocerebrorenal syndrome; Propionic acidemia; |
| | Early preeclampsia; Late-onset preeclampsia; Obesity; |
| | Perillyl alcohol administration for cancer treatment; |
| | Pregnancy; Methylmalonic aciduria mitochondrial |
| | encephalopathy Leigh-like; Long-chain Fatty Acids, |
| | Defect in Transport of; Mitochondrial trifunctional |
| | protein deficiency; Myopathic carnitine deficiency |
| leucine | Periodontal disease; Colorectal cancer; Pancreatic |
| | cancer; Eosinophilic esophagitis; Rheumatoid arthritis; |
| | Ulcerative colitis; Crohn's disease; Branched-chain |
| | Keto Acid Dehydrogenase Kinase Deficiency; |
| | Leukemia; Dihydrolipoamide Dehydrogenase |
| | Deficiency; Fumarase deficiency; Lipoyltransferase 1 |
| | Deficiency; Phenylketonuria; Early preeclampsia; Heart |
| | failure; Late-onset preeclampsia; Epilepsy; Epilepsy, |
| | early-onset, vitamin B6-dependent; Perillyl alcohol |
| | administration for cancer treatment; Pregnancy; |
| | Frontotemporal dementia; Lewy body disease; Autism; |
| | Schizophrenia; Irritable bowel syndrome |
| leucyl-alanine | Colorectal cancer |
| leucyl-leucine | Colorectal cancer |
| leucyl-proline | N/A |
| leucyl-valine | Colorectal cancer; Obesity |
| l-homocysteic acid | N/A |
| lipoamide | N/A |
| lithocholic acid glycine conjugate | N/A |
| l-octanoylcarnitine | Colorectal cancer; Ulcerative colitis; Celiac disease; |
| | Crohn's disease; Glutaric aciduria II; Very Long Chain |
| | Acyl-CoA Dehydrogenase Deficiency; Obesity; |
| | Pregnancy |
| l-phenylalanyl-l-proline | N/A |
| l-theanine | N/A |
| l-thyronine | Amyotrophic lateral sclerosis; Chronic kidney disease; |
| | Hyperthyroidism |
| lumichrome | N/A |
| lysyl-leucine | Colorectal cancer |
| lysyl-valine | Colorectal cancer |
| maltotriose | Peritoneal dialysis |
| mannose 6-phosphate | Colorectal cancer |
| methionine | Colorectal cancer; Eosinophilic esophagitis; |
| | Rheumatoid arthritis; Ulcerative colitis; Celiac disease; |
| | Crohn's disease; Homocystinuria-megaloblastic anemia |
| | due to defect in cobalamin metabolism, cblG |
| | complementation type; Leukemia; Adenosine kinase |
| | deficiency; Citrullinemia type II, neonatal-onset; |
| | Cobalamin F disease (cblF); Fumarase deficiency; |
| | Glycine N-methyltransferase deficiency; |
| | Homocystinuria; Hypermethioninemia; Methionine |
| | adenosyltransferase deficiency; |
| | Methylenetetrahydrofolate reductase deficiency; |
| | Tyrosinemia; Early preeclampsia; Heart failure; Late- |
| | onset preeclampsia; Epilepsy; Obesity; Pregnancy; |
| | Autism; Schizophrenia; Diverticular disease; Irritable |
| | bowel syndrome |
| methionine sulfone | N/A |
| methionyl-glycine | N/A |
| methyl 4-aminobutyrate | N/A |
| methyl galactoside | N/A |
| methylguanidine | Pancreatic cancer; Eosinophilic esophagitis; Uremia; |
| | Chronic renal failure |
| methylthioadenosine | Colorectal cancer |
| mevalolactone | N/A |
| monomethylglutarate | Eosinophilic esophagitis |
| muramic acid | N/A |
| n2-gamma-glutamylglutamine | Crohn's disease; Hyperammonemia; Iron deficiency; |
| | Schizophrenia |
| n6-acetyl-l-lysine | Colorectal cancer; Eosinophilic esophagitis |
| n-acetylalanine | Colorectal cancer; Prostate cancer |
| n-acetylasparagine | N/A |
| n-acetylaspartate | Canavan disease; Schizophrenia; Obesity; Colorectal |
| | cancer; Eosinophilic esophagitis |
| n-acetyl-beta-alanine | N/A |
| n-acetylcysteine | N/A |
| n-acetylgalactosamine | Colorectal cancer |
| n-acetylglutamate | Colorectal cancer; Prostate cancer; Eosinophilic |
| | esophagitis |
| n-acetylglycine | Colorectal cancer; Aminoacylase I deficiency |
| n-acetylhistamine | N/A |
| n-acetylisoleucine | Colorectal cancer |
| n-acetylleucine | Colorectal cancer |
| n-acetyl-l-glutamic acid | Colorectal cancer; Prostate cancer; Eosinophilic |
| | esophagitis |
| n-acetyl-l-methionine | Colorectal cancer |
| n-acetyl-l-tyrosine | Autosomal dominant polycystic kidney disease; |
| | Colorectal cancer; Aromatic L-amino acid |
| | decarboxylase deficiency; Tyrosinemia I; Preterm birth |
| n-acetylmethionine | Colorectal cancer |
| n-acetylmuramate | Colorectal cancer |
| n-acetylneuraminate | Colorectal cancer; Ulcerative colitis; Salla disease; |
| | Sialidosis, normosomatic type; Irritable bowel |
| | syndrome |
| n-acetylphenylalanine | Colorectal cancer |
| n-acetylproline | Colorectal cancer |
| n-acetylputrescine | Colorectal cancer; Leukemia |
| n-acetylserine | Aminoacylase I deficiency |
| n-acetylthreonine | Colorectal cancer |
| n-acetyltryptophan | Colorectal cancer |
| n-acetylvaline | Colorectal cancer |
| n-alpha-acetyllysine | Colorectal cancer |
| n-butyrylglycine | Colorectal cancer; Propionic acidemia; Short Chain |
| | Acyl-Coa Dehydrogenase Deficiency; Ethylmalonic |
| | encephalopathy |
| n-carboxyethyl-gamma- | N/A |
| aminobutyric acid | |
| n-formylphenylalanine | N/A |
| nicotinamide | Colorectal cancer; Ulcerative colitis; Crohn's disease; |
| | Uremia; Diverticular disease |
| nicotinamide mononucleotide | Sodium nitrate consumption |
| nicotinate | Colorectal cancer; Ulcerative colitis; Crohn's disease; |
| | Alcoholism |
| nicotinuric acid | Autosomal dominant polycystic kidney disease |
| n-methyl-alanine | N/A |
| n-methylaspartate | N/A |
| n-methyl-l-proline | Colorectal cancer |
| n-methylnicotinamide | Diabetes mellitus type 2 |
| n-methylphenylalanine | Colorectal cancer |
| norleucine | Colorectal cancer |
| normetanephrine | Pheochromocytoma; Ulcerative colitis; Crohn's disease; |
| | Essential hypertension; Amphetamine psychosis; |
| | Aromatic L-amino acid decarboxylase deficiency; |
| | Brunner Syndrome |
| nutriacholic acid | Colorectal cancer; Hepatocellular carcinoma |
| o-acetylcarnitine | Colorectal cancer; Eosinophilic esophagitis; Ulcerative |
| | colitis; Crohn's disease; 3-Hydroxyacyl-CoA |
| | dehydrogenase deficiency; Short-chain L-3- |
| | hydroxyacyl-CoA dehydrogenase deficiency; Very |
| | Long Chain Acyl-CoA Dehydrogenase Deficiency; |
| | Obesity |
| o-phosphoethanolamine | Colorectal cancer; Eosinophilic esophagitis; Ulcerative |
| | colitis; Crohn's disease; Odontohypophosphatasia; |
| | Traumatic brain injury |
| o-phosphoserine | Eosinophilic esophagitis |
| ophthalmate | N/A |
| o-succinyl-homoserine | N/A |
| pantothenate | Colorectal cancer; Eosinophilic esophagitis; Ulcerative |
| | colitis; Crohn's disease; Alcoholism; Obesity; Irritable |
| | bowel syndrome |
| phenylacetylglycine | Eosinophilic esophagitis; Heart failure |
| phenylalanine | Myocardial infarction; Periodontal disease; Colorectal |
| | cancer; Pancreatic cancer; Eosinophilic esophagitis; |
| | Rheumatoid arthritis; Hypothyroidism; Ulcerative |
| | colitis; Crohn's disease; Leukemia; 6- |
| | Pyruvoyltetrahydropterin synthase deficiency; Gout; |
| | Guanosine triphosphate cyclohydrolase deficiency; |
| | Phenylketonuria; Pterin-4a carbinolamine dehydratase |
| | deficiency; Early preeclampsia; Late-onset |
| | preeclampsia; Alzheimer's disease; Epilepsy; Obesity; |
| | Dengue fever; Perillyl alcohol administration for cancer |
| | treatment; Pregnancy; Viral infection; Frontotemporal |
| | dementia; Lewy body disease; Autism; Schizophrenia; |
| | Bacterial infections; Irritable bowel syndrome |
| phenylalanylalanine | Colorectal cancer |
| phenylethanolamine | Cirrhosis |
| phosphoserine | Eosinophilic esophagitis |
| picolinoylglycine | N/A |
| pipecolate | Uremia; Hepatic encephalopathy; |
| | Adrenoleukodystrophy; Peroxisomal biogenesis defect |
| piperine | Colorectal cancer |
| p-octopamine | Hypertension |
| proline | Autosomal dominant polycystic kidney disease; |
| | Autosomal dominant polycystic kidney disease; |
| | Periodontal disease; Colorectal cancer; Pancreatic |
| | cancer; Eosinophilic esophagitis; Rheumatoid arthritis; |
| | Ulcerative colitis; Celiac disease; Crohn's disease; |
| | Hemodialysis; Hyperprolinemia, type I; |
| | Hyperprolinemia, type II; Dicarboxylic aminoaciduria; |
| | Dihydrolipoamide Dehydrogenase Deficiency; |
| | Glutathione synthetase deficiency; Iminoglycinuria; |
| | Lipoyltransferase 1 Deficiency; Mitochondrial pyruvate |
| | carrier deficiency; Pyruvate carboxylase deficiency; |
| | Early preeclampsia; Late-onset preeclampsia; |
| | Alzheimer's disease; Obesity; Cutis laxa, autosomal |
| | recessive, type IIIA; Perillyl alcohol administration for |
| | cancer treatment; Pregnancy; Frontotemporal dementia; |
| | Lewy body disease; Autism; Irritable bowel syndrome |
| proline betaine | Colorectal cancer |
| prolyl-alanine | Colorectal cancer |
| prolylglycine | Colorectal cancer |
| propionylcarnitine | Eosinophilic esophagitis; Ulcerative colitis; Celiac |
| | disease; Crohn's disease; Propionic acidemia; Obesity |
| pseudouridine | Colorectal cancer; Canavan disease |
| purine | N/A |
| pyridoxal | Colorectal cancer; Sickle cell anemia; Epilepsy, early- |
| | onset, vitamin B6-dependent |
| quinate | Colorectal cancer; Prostate cancer |
| quinine | N/A |
| quinoline-4,8-diol | N/A |
| riboflavin | Colorectal cancer; Eosinophilic esophagitis; |
| | Alcoholism; Anorexia nervosa; Brown-Vialetto-Van |
| | Laere Syndrome 1 |
| ribothymidine | Colorectal cancer; Perillyl alcohol administration for |
| | cancer treatment |
| saccharopine | Colorectal cancer |
| s-adenosylhomocysteine | Neurodegenerative disease; Eosinophilic esophagitis; |
| | Adenosine kinase deficiency; Alzheimer's disease; |
| | Parkinson's disease |
| salicylate | Autosomal dominant polycystic kidney disease; |
| | Colorectal cancer; Eosinophilic esophagitis; |
| | Mitochondrial complex I deficiency due to ACAD9 |
| | deficiency; Colorectal cancer |
| salicyluric acid | Colorectal cancer |
| s-allylcysteine | N/A |
| s-carboxymethylcysteine | N/A |
| sebacate | Colorectal cancer; Eosinophilic esophagitis; Ulcerative |
| | colitis; 3-Hydroxy-3-Methylglutaryl-CoA Synthase |
| | Deficiency; Carnitine-acylcarnitine translocase |
| | deficiency; Iron deficiency |
| serine | Autosomal dominant polycystic kidney disease; |
| | Schizophrenia; Autosomal dominant polycystic kidney |
| | disease; Periodontal disease; Colorectal cancer; Lung |
| | Cancer; Pancreatic cancer; Eosinophilic esophagitis; |
| | Ulcerative colitis; Crohn's disease; Refractory |
| | localization-related epilepsy; Leukemia; 3- |
| | Phosphoglycerate dehydrogenase deficiency; Fumarase |
| | deficiency; Neu-Laxova Syndrome 1; Phosphoserine |
| | Aminotransferase Deficiency; Phosphoserine |
| | Phosphatase Deficiency; Sarcosinemia; Serine |
| | deficiency syndrome, infantile; Early preeclampsia; |
| | Heart failure; Late-onset preeclampsia; Alzheimer's |
| | disease; Juvenile myoclonic epilepsy; Obesity; Perillyl |
| | alcohol administration for cancer treatment; Pregnancy; |
| | Frontotemporal dementia; Lewy body disease; Autism; |
| | Schizophrenia; Irritable bowel syndrome |
| s-lactoylglutathione | N/A |
| syringic acid | Colorectal cancer |
| theobromine | Autosomal dominant polycystic kidney disease; |
| | Colorectal cancer; Malaria; Head injury |
| thiamine monophosphate | Alzheimer's disease |
| thioproline | N/A |
| thiopurine s-methylether | N/A |
| thiourea | N/A |
| threoninyl-glutamine | N/A |
| threoninyl-leucine | Colorectal cancer |
| threoninyl-serine | N/A |
| thymidine | Degenerative disc disease; Colorectal cancer; |
| | Eosinophilic esophagitis; Canavan disease; Thymidine |
| | phosphorylase deficiency |
| thymidine-monophosphate | Colorectal cancer |
| thyrotropin releasing hormone | N/A |
| trans-4-hydroxy-l-proline | Colorectal cancer; Eosinophilic esophagitis; Ulcerative |
| | colitis; Crohn's disease; Hemodialysis; |
| | Hydroxyprolinemia; Iminoglycinuria; Alzheimer's |
| | disease |
| trigonelline | Autosomal dominant polycystic kidney disease; |
| | Colorectal cancer; Eosinophilic esophagitis |
| tryptophan | Periodontal disease; Colorectal cancer; Ovarian cancer; |
| | Pancreatic cancer; Eosinophilic esophagitis; |
| | Rheumatoid arthritis; Hypothyroidism; Ulcerative |
| | colitis; Celiac disease; Crohn's disease; Leukemia; |
| | Hartnup disease; Nicotinamide Adenine Dinucleotide |
| | Deficiency; Tryptophanuria with dwarfism; |
| | Alzheimer's disease; Epilepsy; Friedreich's ataxia; |
| | Hereditary spastic paraplegia; Obesity; Cachexia; |
| | Olivopontocerebral atrophy; Perillyl alcohol |
| | administration for cancer treatment; Frontotemporal |
| | dementia; Lewy body disease; Autism; Schizophrenia; |
| | Irritable bowel syndrome |
| tyramine | Colorectal cancer; Eosinophilic esophagitis; Ulcerative |
| | colitis; Celiac disease; Crohn's disease; Brunner |
| | Syndrome |
| tyrosine | Colorectal cancer; Pancreatic cancer; Eosinophilic |
| | esophagitis; Hypothyroidism; Ulcerative colitis; |
| | Crohn's disease; Leukemia; Citrullinemia type II, |
| | neonatal-onset; Fumarase deficiency; Hawkinsinuria; |
| | Hypermethioninemia; Tyrosinemia; Tyrosinemia I; |
| | Early preeclampsia; Late-onset preeclampsia; |
| | Alzheimer's disease; Epilepsy; Obesity; Cachexia; |
| | Perillyl alcohol administration for cancer treatment; |
| | Pregnancy; Viral infection; Frontotemporal dementia; |
| | Lewy body disease; Autism; Schizophrenia; Irritable |
| | bowel syndrome; Myocardial infarction |
| undecanedioic acid | Colorectal cancer; Ulcerative colitis; Iron deficiency |
| uracil 5-carboxylate | N/A |
| urate | Degenerative disc disease; Colorectal cancer; Stomach |
| | cancer; Diabetes mellitus type 2; Fructose intolerance, |
| | hereditary; ATIC deficiency; Canavan disease; |
| | congenital disorder of glycosylation CDG-Ia; D-Lactic |
| | Acidosis; Fructose-1,6-diphosphatase deficiency; Gout; |
| | Impaired glucose tolerance; Lesch-Nyhan syndrome; |
| | Long-chain Fatty Acids, Defect in Transport of; |
| | Molybdenium co-factor deficiency; Molybdenum |
| | cofactor deficiency; Nucleotide Depletion Syndrome; |
| | Phosphoribosylpyrophosphate Synthetase |
| | Superactivity; Sulfite oxidase deficiency, ISOLATED; |
| | Uremia; Xanthinuria type 1; Cachexia; Schizophrenia; |
| | Primary hypomagnesemia; Bacterial meningitis; |
| | Cerebral creatine deficiency syndrome 2; Fanconi |
| | Bickel syndrome; Fanconi syndrome |
| ureidopropionate | Beta-ureidopropionase deficiency; Colorectal cancer |
| ureidopropionic acid | Colorectal cancer; Dihydropyrimidinase deficiency |
| uridine | Degenerative disc disease; Colorectal cancer; |
| | Ulcerative colitis; Argininemia; Canavan disease; |
| | Lesch-Nyhan syndrome; Irritable bowel syndrome |
| uridine monophosphate | Alzheimer's disease; Frontotemporal dementia; Lewy |
| | body disease |
| urocanate | Colorectal cancer |
| valerylglycine | Propionic acidemia |
| val-val-val | Colorectal cancer |
| valyl-glutamate | Colorectal cancer |
| valyl-glycine | Colorectal cancer |
| valyl-methionine | Colorectal cancer |
| valyl-serine | Colorectal cancer |
| valyl-tyrosine | Colorectal cancer |
| valyl-valine | Colorectal cancer |
| xanthine | Degenerative disc disease; Colorectal cancer; |
| | Eosinophilic esophagitis; Ulcerative colitis; Crohn's |
| | disease; Adenosine kinase deficiency; Canavan disease; |
| | Lesch-Nyhan syndrome; Molybdenium co-factor |
| | deficiency; Phosphoribosylpyrophosphate Synthetase |
| | Superactivity; Sulfite oxidase deficiency, ISOLATED; |
| | Xanthinuria type 1; Xanthinuria type II; |
| | Hydrocephalus; Cystic fibrosis |

As used herein, the terms “standard” and/or “internal standard” refer to a well-characterized substance of known amount and/or identity (e.g., known molecular weight, electrophoretic mobility profile) that can be added to a sample and both the standard and the molecules in the sample can be characterized on the basis of molecular weight or isoelectric point by electrophoresis. A comparison to the standard then provides a quantitative or semi-quantitative measure of the amount of analyte, such as ATP, present in the sample.

“Contacting,” as used herein, includes bringing together at least two substances in solution or solid phase.

“Mass spectrometry” refers to a method in which a sample is analyzed by generating gas phase ions from the sample, which are then separated according to their mass-to-charge ratio (m/z) and detected. Prior to detection, the sample may be subjected to one or more dimensions of chromatographic separation, for example, one or more dimensions of liquid or size exclusion chromatography.

Samples for use in the disclosed methods can be heterogeneous, containing a variety of components, i.e. different metabolites. Alternatively, the sample can be homogenous, containing one metabolite or essentially one metabolite of multiple charge or molecular weight species. Pre-analysis processing may be performed on the sample prior to detecting the metabolite.

Historically, use of microfluidic-based CE/MS systems have been a challenge for analyzing anionic substrates. However, using the methods described herein, discrimination of anionic, neutral, and positively charged molecules is possible. In some aspects, microchip or microfluidic-based CE/MS systems are used for the analyses. In some aspects, the microchip is surface modified to comprise a substrate. An example of a microchip-based CE system that can be used in tandem with MS is the ZipChip™ (908 Devices, Boston, MA).

In some aspects, the capillary can include a separation matrix, which can be added in an automated fashion by the apparatus and/or system. In some aspects, the sample is loaded onto a stacker matrix prior to separation. The separation matrix, in one aspect, is a size separation matrix, and has similar or substantially the same properties of a polymeric gel, used in conventional electrophoresis techniques. Capillary electrophoresis in the separation matrix is analogous to separation in a polymeric gel, such as a polyacrylamide gel or an agarose gel, where molecules are separated on the basis of the size of the molecules in the sample, by providing a porous passageway through which the molecules can travel. The separation matrix permits the separation of analytes by molecular size because larger molecules will travel more slowly through the matrix than smaller molecules. In some aspects, the one or more capillaries comprise a separation matrix. In some aspects, the sample containing a metabolite is separated or resolved based on molecular weight. In some aspects, the separation matrix comprises a sieving matrix configured to separate proteins by molecular weight. In some embodiments, protein components of a sample are separated by molecular weight and the method is a method of detecting and/or discriminating between size variants of a metabolite and its analogues or degradation products.

In some aspects, the sample containing a metabolite of interest is separated or resolved based on the charge of the components of the sample. In some aspects, metabolite components of a sample are separated by charge and the method is a method of detecting and/or discriminating between charge variants of a metabolite and its analogues or degradation products.

In some aspects, an internal standard can be used to quantitatively detect the metabolite of interest. The internal standard can be a purified form of the metabolite of interest that is distinguishable from the metabolite of interest in some way. The distinguishing characteristic of an internal standard can be any suitable change that can include, but is not limited to, dye labeling, stable isotope enrichment, or modifying the mobility of the standard during the electrophoretic separation so that it is separated from the metabolite of interest.

Virtually any method of loading the sample in the capillary may be performed. For example, the sample can be loaded into one end of the capillary. In some aspects, the sample is loaded into one end of the capillary by hydrodynamic flow. For example, in embodiments wherein the fluid path is a capillary, the sample can be loaded into one end of the capillary by hydrodynamic flow, such that the capillary is used as a micropipette. In some aspects, the sample can be loaded into the capillary by electrophoresis, for example, when the capillary is gel filled and therefore more resistant to hydrodynamic flow.

The capillary can include any microchip structure that allows liquid or dissolved molecules to flow. Thus, the capillary can include any structure known in the art, so long as it is compatible with the methods. In some embodiments, the capillary is a bore or channel through which a liquid or dissolved molecule can flow. In some embodiments, the capillary is a passage in a permeable material in which liquids or dissolved molecules can flow.

The capillary includes any material that allows the separation of the metabolite of interest within the capillary. The capillary includes any convenient material, such as glass, plastic, silicon, fused silica, gel, or the like. In some aspects, the method employs a plurality of capillaries. A plurality of capillaries enables multiple samples to be analyzed simultaneously. In some aspects, the microchips containing the capillary are coated. In other aspects, the microchips are bare glass.

### Disease or Disorder Targets

The methods described herein are useful for detection of metabolites associated with a disease or disorder. In one aspect, the disease or disorder is shown in Table 1. In another aspect, the disease or disorder is associated with increased levels of a nucleotide, for example ATP, and is either an airway inflammation disease, cough, heart disease, eye disease, neurodegenerative disease, psychiatric disease, neuropathic pain, a chronic inflammatory disease, metabolic disease, or cancer.

For example, ATP possesses all the features of an ideal extracellular messenger: (a) is virtually absent in the extracellular space under physiological conditions (estimated concentration 10-100 nmol/L); (b) is stored in very high amounts within the cells (from 5 to 10 mmol/L); (c) is water-soluble and freely diffusible in the extracellular space due to negatively charged phosphate residues; (d) is rapidly degraded by ubiquitous extracellular nucleotidases; (e) ligates specific plasma membrane receptors, a feature that confers specificity to its signaling. These properties allow the generation of an extracellular messenger characterized by (a) very low background noise and thus high signal-to-noise ratio; (b) rapid diffusion through the aqueous tissue interstitium; (c) rapid signal shut-off to avoid overstimulation or receptor desensitization.

A role for eATP has been identified in several, different physiological and pathological conditions an airway inflammation disease, cough, heart disease, eye disease, neurodegenerative disease, psychiatric disease, neuropathic pain, a chronic inflammatory disease, metabolic disease, or cancer. For example, eATP plays an important role in pulmonary physiology, including epithelial ciliary sodium and water transport and mucin secretion. eATP is rapidly degraded to adenosine 5′-diphosphate, adenosine 5′-monophosphate, and adenosine by ectoenzymes, mainly CD39 and CD73. Although the rapid degradation of eATP results in low levels of extracellular ATP in general, specific microenvironmental and pathophysiologic conditions, such as airway inflammation diseases, are associated with elevated local concentration of eATP. ATP also enhances the cough reflex. eATP and P2X2/3R have been implicated in the mechanism of cough in patients with chronic idiopathic cough.

Therefore, in one aspect the present disclosure is directed to methods of detecting an airway inflammation disease, cough, heart disease, eye disease, neurodegenerative disease, psychiatric disease, neuropathic pain, a chronic inflammatory disease, metabolic disease, or cancer in subjects by detecting the presence of ATP and/or its nucleotide analogues or degradation products in a sample. In another aspect, the presence of ATP and/or its nucleotide analogues or degradation products can be used to assess the therapeutic benefit of a compound used to treat airway an airway inflammation disease, cough, heart disease, eye disease, neurodegenerative disease, psychiatric disease, neuropathic pain, a chronic inflammatory disease, metabolic disease, or cancer.

### EXAMPLES

Reference is now made to the following example, which together with the above descriptions illustrate some embodiments of the invention in a non-limiting fashion.

### Materials

LC-MS grade water, methanol, and ammonium hydroxide were purchased from Fisher Scientific (Hampton, NH). Adenosine ¹³C5 was obtained from Cambridge Isotope Laboratories, Inc. (Tewksbury, MA). Ammonium formate, adenosine-¹³C10,¹⁵N5 5′-monophosphate, adenosine-¹⁵N5 5′-diphosphate, adenosine-¹³C10,¹⁵N5 5′-triphosphate and the correspondent unlabeled ATP, ADP, AMP, and adenosine standards were purchased from Millipore Sigma (Burlington, MA). Strata™ X-AW 33 um Polymeric Weak Anion solid phase extraction (SPE) columns were purchased from Phenomenex (Torrance, CA).

### Specimen Collection

Human blood and plasma blood-derived samples were collected through the Research Specimen Collection Program, available at AstraZeneca in Gaithersburg, MD, following the participant's informed consent signature and enrollment. HeLa cells and Naïve Wistar rat plasma and BALF (bronchoalveolar lavage fluid) samples were provided by AstraZeneca collaborators. EDTA was used while collecting biological samples to prevent ATP hydrolysis.

### Sample Preparation

Metabolites, including ATP and its breakdown products, were extracted using 100% methanol, spiked with internal standards to yield a final concentration of 1 μM, at a ratio of 1:20 for blood, 1:8 for plasma, 1:3 for BALF, 1000 cells: 100 μL for HeLa cells, respectively. To allow protein precipitation, a few cycles of vortexing and sonication on ice were performed before final centrifugation at 14,000×g, for 10 minutes at 4° C. The collected supernatants were concentrated by speed vac, set at room temperature, and reconstituted in 25 μL of LC-MS grade water prior to CE-MS analysis. Specifically, for BALF samples, an additional desalting step was followed using polymeric weak anion SPE columns: concentrated BALF samples were diluted 1:1 with acidified (pH 4) ammonium formate 10 mM before passing through an SPE column, previously activated with methanol and equilibrated with acidified ammonium formate 10 mM. After washing the column with acidified ammonium formate 10 mM first (Wash 1) and then methanol (Wash 2), metabolites were eluted with 5% ammonium hydroxide in methanol. The eluted and Wash 2 fractions were combined and concentrated by speed vac and then resuspended in 25 μL of LC-MS grade water prior to CE-MS analysis. A stock solution of unlabeled ATP and its breakdown products was serially diluted in LC-MS grade water to prepare different calibration curve points and three quality control (QC) solutions. The preparation of curve points and QC followed the same procedure as described for the biological samples.

### ZipChip Consumables

HRB chip from the Cartridge Package (5 pack) with cat #810-0023 and BGE from the Native Antibodies kit (cat #850-00048) were purchased from 908 Devices (Boston, MA, USA). The Native Antibodies BGE, used to prime the autosampler, comes at a pH of 5.5. For this application, it was required to achieve pH of ˜8.6 by adjusting it with ammonium hydroxide.

### CE Method

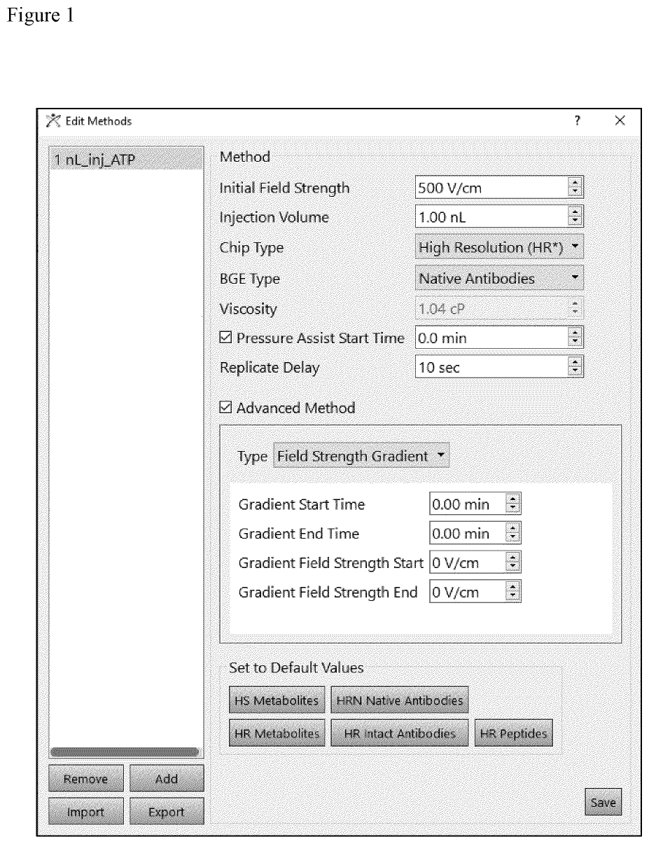

All analyses were performed using a ZipChip™ device and autosampler from 908 Devices (Boston, MA). The microfluidic chip settings were: initial field strength 500 V/cm, injection volume 1.00 nL, viscosity 1.04 cP, pressure assist start time 0 min, replicate delay 10 sec. FIG. 1 summarizes the capillary electrophoresis parameters used.

### MS Method

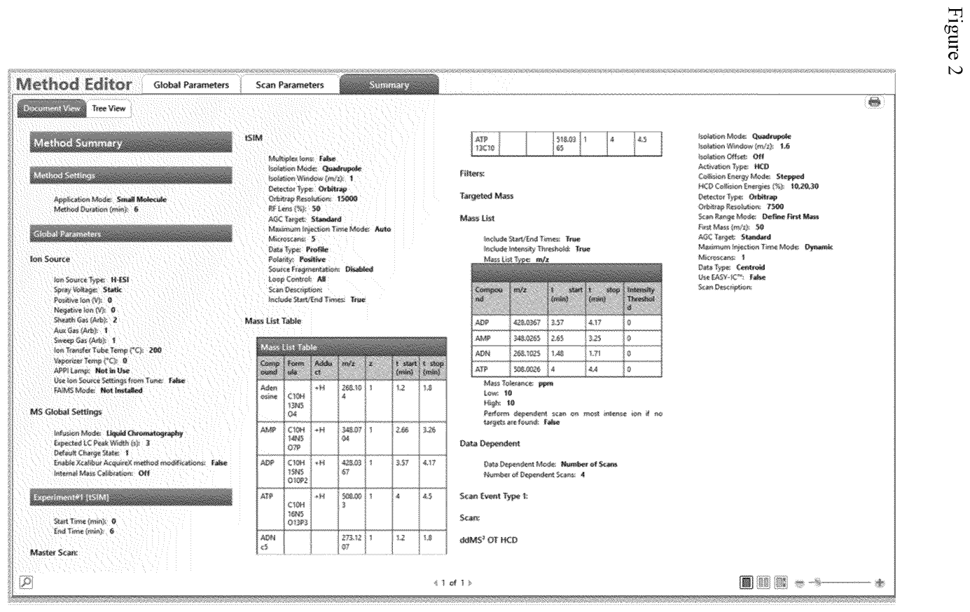

This protocol was demonstrated using a Thermo Scientific IDX. FIG. 2 summarizes the mass spectrometer parameters used. Optimal setting for other mass spectrometers can be different. Data acquisition was accomplished through the Thermo Xcalibur™ tune page which was triggered by the ZipChip software. Analysis run time lasted for 6 minutes.

### Data Analysis

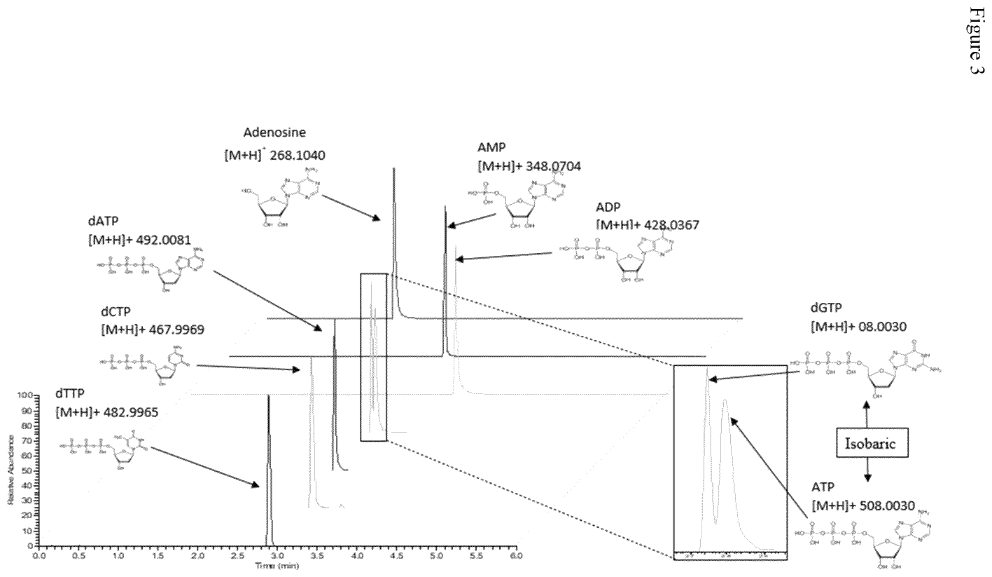

Data obtained from the targeted analysis was processed using Thermo Xcalibur™ Quan Browser software. FIG. 3 shows an electropherogram of ATP, ADP, AMP, Adenosine, dATP, dGTP, dCTP, and dTTP using the above-described methods.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for detecting a metabolite of interest in a sample, comprising:
(a) contacting a sample comprising one or more metabolites of interest with an uncoated capillary electrophoresis (CE) platform;
(b) separating the metabolites by molecular weight and/or charge in one or more capillaries using CE;
(c) eluting the metabolite from the one or more capillaries; and
(d) detecting the eluted metabolite by mass spectrometry analysis.

2. A method for detecting a metabolite of interest in a sample, comprising:
(a) contacting a sample comprising one or more metabolites of interest with a capillary electrophoresis (CE) platform having a chemically-modified surface;
(b) separating the metabolites by molecular weight and/or charge in one or more capillaries using CE;
(c) eluting the metabolite from the one or more capillaries; and
(d) detecting the eluted metabolite by mass spectrometry analysis.

3. The method of claim 1 or claim 2, wherein the CE platform is a microchip-based system.

4. The method of claim 3, wherein the microchip-based CE platform integrates electrophoretic separation and electrospray ionization into a mass spectrometer.

5. The method of any one of claims 1-4, wherein the metabolite of interest is listed in Table 1.

6. The method of any one of claims 1-5, wherein the metabolite of interest is an anionic metabolite.

7. The method of any one of claims 1-6, wherein the metabolite is a nucleotide, nucleotide analog, or degradation product.

8. The method of any one of claims 1-7, wherein the metabolite of interest is adenosine 5′-triphosphate (ATP).

9. The method of any one of claims 1-8, wherein the sample is a blood, plasma, cell, or lavage sample.

10. The method of claim 9, wherein the sample is a bronchoalveolar lavage fluid (BALF).

11. The method of any one of claims 1-10, wherein the sample comprises a chelating agent.

12. The method of claim 11, wherein the chelating agent is ethylenediaminetetraacetic acid (EDTA).

13. A method of diagnosing a disease or disorder associated with aberrant nucleotide-dependent signaling in a subject, comprising:
(a) contacting a sample from the subject with microchip-based capillary electrophoresis (CE) platform;
(b) separating the adenosine 5′-triphosphate (ATP), ATP analogues and/or degradation products by molecular weight and/or charge in one or more capillaries using CE; and
(c) eluting the ATP, ATP analogues and/or degradation products from the one or more capillaries; and
(d) detecting the eluted ATP, ATP analogues and/or degradation products by mass spectrometry analysis;
wherein the microchip-based CE platform integrates electrophoretic separation and electrospray ionization into a mass spectrometer; and
wherein the presence of ATP, ATP analogues and/or degradation products is indicative of a disease or disorder associated with aberrant nucleotide-dependent signaling in the subject.

14. A method of monitoring the therapeutic benefit of a compound on a disease or disorder associated with aberrant nucleotide-dependent signaling in a subject treated with the compound, comprising:
(a) contacting a sample from the subject with microchip-based capillary electrophoresis (CE) platform;
(b) separating the ATP, ATP analogues and/or degradation products by molecular weight and/or charge in one or more capillaries using CE; and
(c) eluting the ATP, ATP analogues and/or degradation products from the one or more capillaries; and
(d) detecting the eluted ATP, ATP analogues and/or degradation products by mass spectrometry analysis;
wherein the microchip-based CE platform integrates electrophoretic separation and electrospray ionization into a mass spectrometer, and
wherein the presence of ATP, ATP analogues and/or degradation products is indicative of a disease or disorder associated with aberrant nucleotide-dependent signaling in the subject.

15. The method of claim 13 or 14, wherein the disease or disorder is associated with increased levels of extracellular ATP (eATP).

16. The method of any one of claims 13-15, wherein the disease or disorder is an airway inflammation disease, cough, heart disease, eye disease, neurodegenerative disease, psychiatric disease, neuropathic pain, a chronic inflammatory disease, metabolic disease, or cancer.

17. The method of claim 16, wherein the cough is chronic idiopathic cough.

18. The method of claim 16, wherein the chronic inflammatory disease is systemic lupus erythematosus or Crohn's disease.

19. The method of any one of claims 13-18, wherein the sample is a blood, plasma, cell, or lavage sample.

20. The method of claim 19, wherein the sample is a BALF.

21. The method of any one of claims 13-20, wherein the sample comprises a chelating agent.

22. The method of claim 21, wherein the chelating agent is ethylenediaminetetraacetic acid (EDTA).

23. The method of any one of claims 13-22, wherein the microchip is a ZipChip™.

24. The method of any one of claims 3-23, wherein the microchip comprises a chemically-modified surface.

25. The method of any one of claims 3-23, wherein the microchip does not comprise a surface modification.

26. The method of any one of claims 1-25, further comprising adjusting the pH of the background electrolyte (BGE) relative to the metabolite of interest prior to mass spectrometry analysis.
